(19) United States
(12) Patent Application Publication
(10) Pub. No.: US 2025/0087359 A1
(43) Pub. Date: Mar. 13, 2025

(54) Neonate Evaluator and Care Manager

(71) Applicant: Innovative Veterinary Practices, Lomira, WI (US)

(72) Inventors: Marthina Greer, Lomira, WI (US); Daniel Griffiths, Lomira, WI (US)

(73) Assignee: Innovative Veterinary Practices, Lomira, WI (US)

(21) Appl. No.: 18/367,088

(22) Filed: Sep. 12, 2023

(51) Int. Cl. G16H 50/30; G16H 20/00

(52) U.S. Cl. CPC G16H 50/30; G16H 20/00

(57) ABSTRACT

A neonate evaluator that is able to automatically evaluate neonate health is described. The neonate evaluator may be able to generate various health metrics or scores related to the neonate evaluation. The neonate evaluator may be able to provide care recommendations based on the health metrics or scores. The neonate evaluator may utilize machine learning to optimize neonate evaluation and care.

### BACKGROUND

Pet breeders, ranchers, veterinarians, and/or other users wish to accurately assess and manage neonate health.

Therefore there exists a need for ways to collect and analyze neonate data in order to generate one or more health score metrics and to identify relevant and effective treatment options.

### BRIEF DESCRIPTION OF THE SEVERAL VIEWS OF THE DRAWING

The novel features of the disclosure are set forth in the appended claims. However, for purpose of explanation, several embodiments are illustrated in the following drawings.

FIG. 1 illustrates an example overview of one or more embodiments described herein, in which a neonate evaluator collects and analyzes neonate data and provides a health score and care recommendations;

FIG. 2 illustrates a schematic block diagram of an exemplary neonate evaluator of one or more embodiments described herein;

FIG. 3 illustrates a schematic block diagram of an exemplary environment of one or more embodiments described herein;

FIG. 4 illustrates a data structure diagram of exemplary data structures used by the neonate evaluator of one or more embodiments described herein;

FIG. 5 illustrates a lookup table associated with an exemplary evaluation plan utilized by the neonate evaluator of one or more embodiments described herein;

FIG. 6A illustrates a lookup table associated with exemplary care information utilized by the neonate evaluator of one or more embodiments described herein;

FIG. 6B illustrates a lookup table associated with exemplary care recommendations utilized by the neonate evaluator of one or more embodiments described herein;

FIG. 7 illustrates a flow chart of an exemplary process that manages data collection for neonate evaluation;

FIG. 8 illustrates a flow chart of an exemplary process that generates a health score and care recommendations;

FIG. 9 illustrates a flow chart of an exemplary process that maps sensor data to evaluation parameters when generating a health score based on collected data;

FIG. 10 illustrates a flow chart of an exemplary process that identifies relevant health care recommendations;

FIG. 11 illustrates a flow chart of an exemplary process that manages equipment used to evaluate neonates;

FIG. 12 illustrates a flow chart of an exemplary process that applies machine learning to neonate evaluation and care recommendations; and

FIG. 13 illustrates a schematic block diagram of one or more exemplary devices used to implement various embodiments.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide a neonate evaluator that is able to automatically evaluate neonate health. The neonate evaluator may be able to generate various health metrics or scores related to the neonate evaluation. The neonate evaluator may be able to provide care recommendations based on the health metrics or scores. The neonate evaluator may utilize machine learning to optimize neonate evaluation and care.

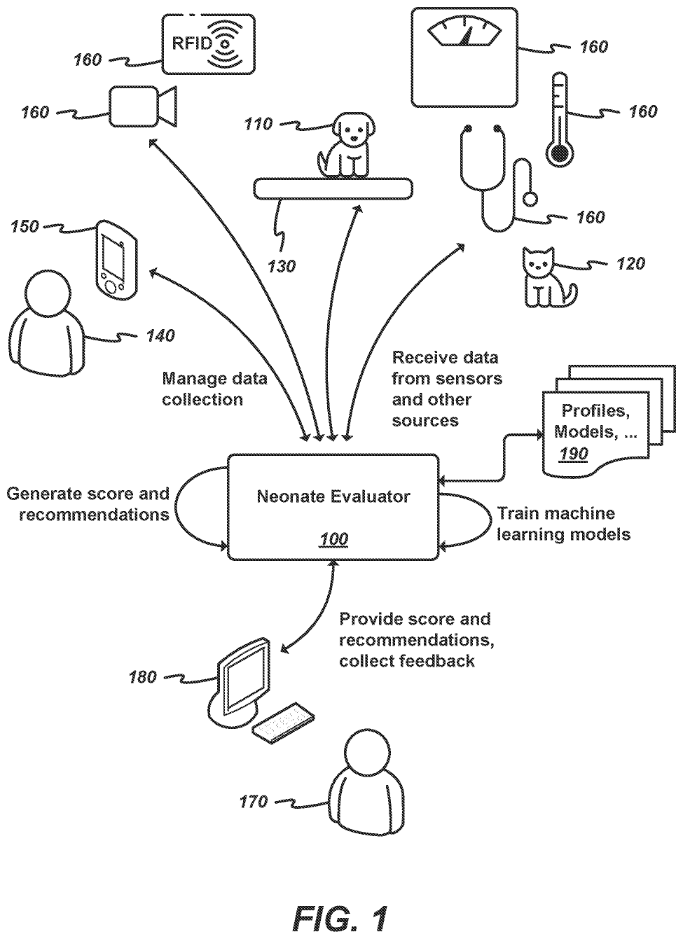

FIG. 1 illustrates an example overview of one or more embodiments described herein, in which an exemplary neonate evaluator 100 collects and analyzes neonate data and provides a health score and care recommendations. Neonate evaluator 100 may be an electronic computing device or system and may be, include, manage, utilize, and/or otherwise be associated with various electro-mechanical devices and/or systems. Neonate evaluator 100 may include various user interface (UI) features, such as touchscreens, buttons, keypads, etc.

Subject neonates 110 and 120 may be newborn or infant animals that may require specialized evaluation and/or care during, for example, the first two to four weeks after birth. Example neonate animals may include puppies, kittens, and/or other mammals (e.g., calves, lambs, etc.). In some embodiments, neonates may include other types of animals, such as birds, reptiles, amphibians, and/or fish.

Evaluation facility 130 may include an appropriate area for neonate evaluation, such as a building, examination room, and/or other appropriate areas depending on the neonate attributes. In some embodiments, neonate evaluator 100 may include evaluation facility 130 as a sub-element. In this example, evaluation facility 130 is represented as a platform or other area that may be used for neonate evaluation.

In this example, user 140 may be a local user that is able to interact with the subject neonate 110 or 120 and is able to interact with neonate evaluator 100, either via a UI feature of neonate evaluator 100 or via a resource such as user device 150. Evaluation facility 130 may provide, include, utilize, manage, and/or otherwise be associated with various evaluation sensors 160, fixtures (e.g., a kennel, cage, leash or harness attachment, etc.), user devices 150, and/or other appropriate devices or systems (e.g., a network gateway, environmental sensors or control systems, etc.).

In some embodiments, evaluation facility 130 may be, include, and/or otherwise be associated with one or more automated entities such as robotic devices or systems, conveyors, etc. Neonate evaluator 100 may direct or control the operations of evaluation facility 130. For instance, a set of messages may be sent from the neonate evaluator 100 to the evaluation facility 130 in order to provide a neonate 110 for evaluation. For instance, a next neonate 110 may be moved along a conveyor to an evaluation area associated with evaluation facility 130.

In this example, neonate 110 may be associated with a fixture or specified evaluation area of facility 110, while neonate 120 may be monitored via implantable or wearable sensors 160 and/or sensors 160 that may capture neonate data at regular intervals or under other appropriate conditions (e.g., a camera 160 may capture image data as a neonate sleeps, eats, moves about, etc.).

User 140 may be a technician, veterinarian, and/or other entity that may be associated with evaluation of a neonate 110 or 120. In some embodiments, user 140 may be, include, and/or otherwise be associated with one or more automated entities such as robotic devices or systems, conveyors, etc. Neonate evaluator 100 may direct or control the operations of user 140. For instance, instructions may be provided via a UI of user device 150 or a set of messages may be sent to a robotic or automated user 140.

User device 150 may be a computing device such as a smartphone, tablet, personal computer (PC), wearable device, etc. User device 150 may include various UI elements (e.g., touchscreen, display, keypad, buttons, joystick, haptic elements, etc.). User device 150 may be able to communicate across various channels, such as wired, wireless, via one or more networks, etc. User device 150 may include, provide, manage, utilize, and/or otherwise interact with various sensors 160. For instance, image data may be captured via a camera of a smartphone 150. As another example, breathing sounds may be recorded via a microphone of user device 150.

Manually collected information may be received via a resource such as user device 150 or a UI feature of neonate evaluator 100. For instance, neonate evaluator 100 may provide prompts to user 140 (e.g., “measure temperature using rectal thermometer”) and measured data (e.g., a manual thermometer reading) may be entered via the UI feature of neonate evaluator 100 or user device 150. Subjective information may be collected in a similar manner. For instance, various choices may be provided for energy level (“lethargic”, “normal”, “hyperactive”, etc.). Other collected information may include additional notes or feedback.

Sensors 160 may be, include, and/or otherwise utilize various components that may measure physical and/or physiological attributes of a neonate 110 or 120. For example, sensors 160 may include cameras, microphones, radio frequency identification (RFID) sensors, scales, thermometers, stethoscopes, heart rate monitors, glucometers, temperature sensors, etc. In some cases, sensors 160 may utilize, include, and/or otherwise be associated with physical reference elements such as rulers, color charts, etc. Each sensor 160 may be able to communicate across various channels, such as wired, wireless, via one or more networks, etc. Sensors 160 may be associated with facility 130, user 130, and/or other appropriate resources (e.g., wearable and/or implanted sensors 160 associated with a particular neonate 110).

User 170 may be similar to user 140. In this example, user 170 may interact with neonate evaluator 100 at a different place or time than user 140. For instance, user 170 may review collected data from a different site than facility 130 via a network connection. User device 180 may be similar to user device 150.

Neonate evaluator 100 may be able to control or direct the operations of sensors 160. For instance, neonate evaluator 160 may send a set of messages to a camera sensor 160 to direct the sensor 160 to collect image data, video, audio data, etc. As another example, neonate evaluator may send a request for a current measurement from a sensor 160 such as a bodyweight scale.

Neonate evaluator 100 may include, direct, and/or otherwise interact with various positioning or other control elements associated with sensors 160, such as robotic arms, gantries, transports, lifts, etc. In some embodiments, facility 130 may provide, include, and/or be associated with such positioning elements. Such positioning elements may be able to manipulate sensor position, neonate position, environmental conditions, and/or otherwise control the evaluation environment associated with facility 130. For instance, neonate evaluator 100 may be able to automatically position an infrared laser thermometer sensor 160 using data captured via a camera sensor 160. As another example, neonate evaluator 100 may be able to automatically position an evaluation platform or support based on the measured size of a neonate 110 (e.g., as determined via a camera sensor 160 and ruler, as determined via a bodyweight scale, etc.) such that the neonate 110 is properly positioned for evaluation.

Some sensors 160 may be used to evaluate multiple neonates 110. For example, multiple neonates 110 may be evaluated at a particular area of facility 130 associated with an infrared laser temperature sensor 160 or bodyweight scale sensor 160. Some sensors 160 may be associated with a particular neonate 110 or 120, such as a wearable hear rate monitor, implantable microchip, etc.

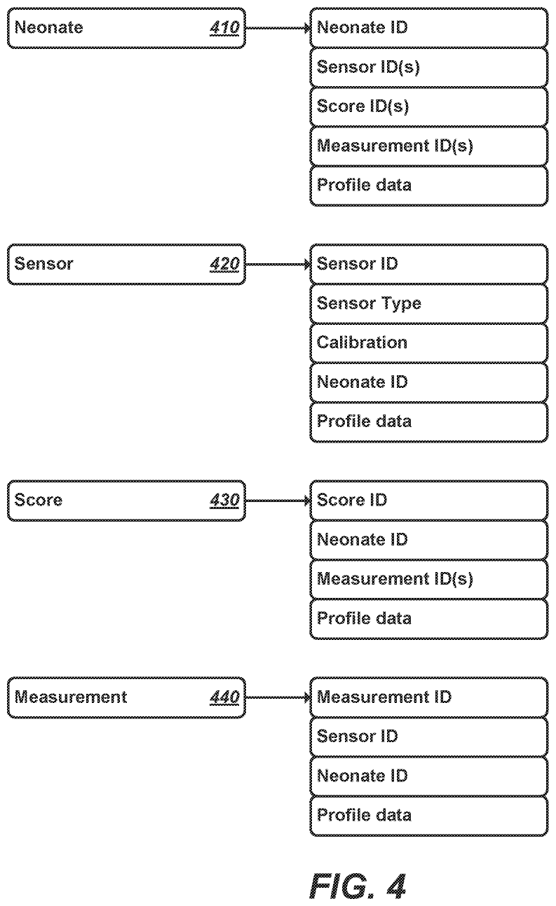

Profiles, models, and/or other data elements 190 (e.g., machine learning models) may be generated, managed, and/or otherwise utilized by neonate evaluator 100. Various example data elements are described in reference to FIG. 4 below.

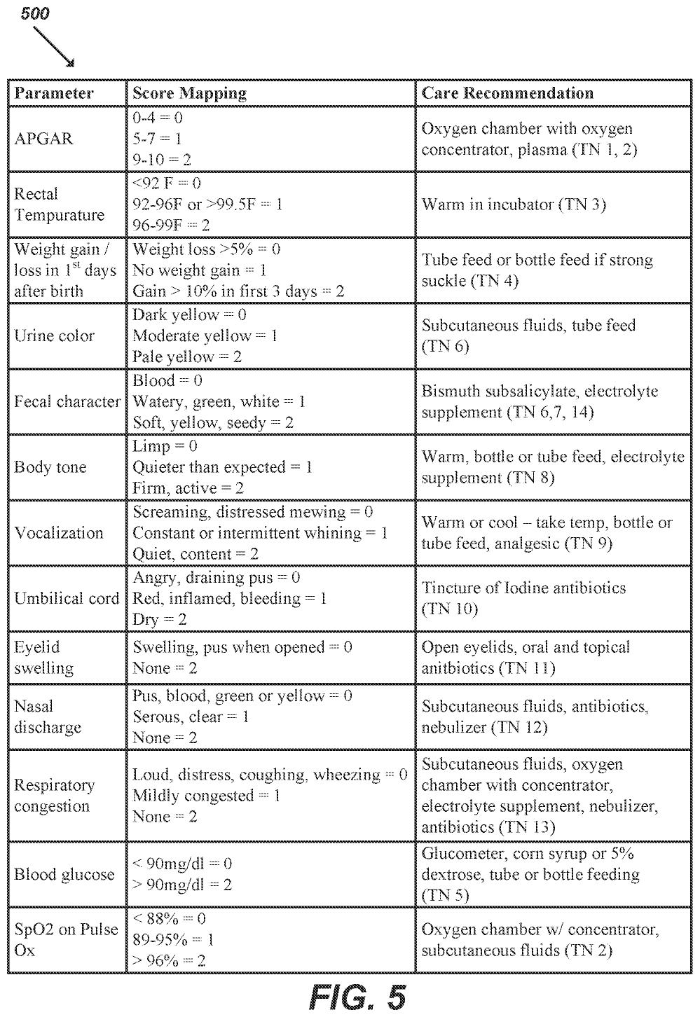

Returning to FIG. 1, neonate evaluator 100 may be able to determine information related to neonate 110, such as by using an RFID scanner sensor 160 to scan for RFID microchips, using a camera sensor 160 to capture and analyze image data to identify neonate attributes (e.g., identity, type, breed, identity, size, etc.), receiving inputs from user 140 via user device 150, and/or other appropriate ways. Based on the neonate attributes, profile or other data 190, and/or other relevant factors (e.g., capabilities of facility 130, user preferences, etc.), the neonate evaluator 100 may identify or generate an evaluation plan (e.g., a listing of measurements to be taken). One example evaluation plan and associated score and care recommendation mapping is described in reference to FIG. 5 below.

As shown in FIG. 1, neonate evaluator 100 may manage data collection. The evaluation plan may be provided to user 140 via user device 150, a UI feature of neonate evaluator 100 and/or other appropriate ways. Data may be collected in various appropriate ways. For instance, neonate evaluator 100 may automatically retrieve data from available sensors 160, as indicated by the evaluation plan. As another example, instructions may be provided to user 140 via user device 150 or a UI feature of neonate evaluator 100 indicating a next measurement type and/or allowing entry of measured values. As another example, a next measurement type may be provided to user 140 via user device 150 or a UI feature of neonate evaluator 100 and neonate evaluator 100 may wait for confirmation that the measurement sensor 160 is properly positioned or other evaluation criteria are satisfied before automatically collecting a reading from the sensor 160.

Neonate evaluator 100 may receive data from the sensors 160, user device(s) 150 and/or UI features (e.g., data received from user 140), and/or other appropriate sources. Neonate evaluator 100 may automatically determine measurement type, scale, and/or other relevant measurement information related to received data. For instance, each sensor 160 may transmit an identifier that may be used to retrieve a sensor profile 190 indicating various attributes of the sensor 160 (e.g., type, scale, calibration data, accuracy, etc.). As another example, neonate evaluator 100 may utilize one or more machine learning models 190 and/or lookup tables 190 to automatically identify attributes of the sensor 160 by analyzing the received data (e.g., by matching portions of received data to data portions of a lookup table 190, using a machine learning model to identify an attribute, etc.). Measurement attributes may be determined based on data received from a user device 150 or UI of the neonate evaluator 100.

The evaluation plan may include a listing of measurements or other evaluation data. The evaluation plan may be associated with various scoring metrics. For instance, each measurement in the listing may be associated with a weighting or other averaging feature. As another example, the evaluation plan may be associated with score mapping information that allows raw measurements to be normalized and/or otherwise manipulated. For example, an evaluation plan for a kitten may include fifteen equally weighted elements, with each element scored from zero to two points to generate a combined score of zero to thirty points. Some measurements may include multiple components and/or data received from multiple sensors 160 and/or types of sensors 160 (e.g., a “size” measurement may include or be based on photographic information of the neonate next to a ruler, bodyweight measurements, and/or other appropriate data).

The evaluation plan may be updated based on received measurements. For instance, if a received measurement exceeds a threshold value, additional measurements and/or types of measurements may be taken and/or recommended. For example, if a body temperature measured by an infrared laser sensor 160 is above a maximum threshold or below a minimum threshold, a manual body temperature measurement (e.g., via rectal thermometer) may be indicated or recommended.

Neonate evaluator may generate a score for each element in the evaluation plan. Continuing the kitten example, each measured value (or set of values) may be mapped to a score of zero to two for each element. The mapped scores may be summed or otherwise combined (e.g., via averaging) to generate an overall score. In the kitten example, each parameter score may be equally weighted.

Recommendations may include general recommendations based on overall score. For example, a score below a lower threshold may indicate that immediate and urgent veterinary care is needed. As another example, a score above the lower threshold and below an upper threshold may indicate that veterinary intervention should be provided when next available. A score above the upper threshold may indicate that no intervention is needed.

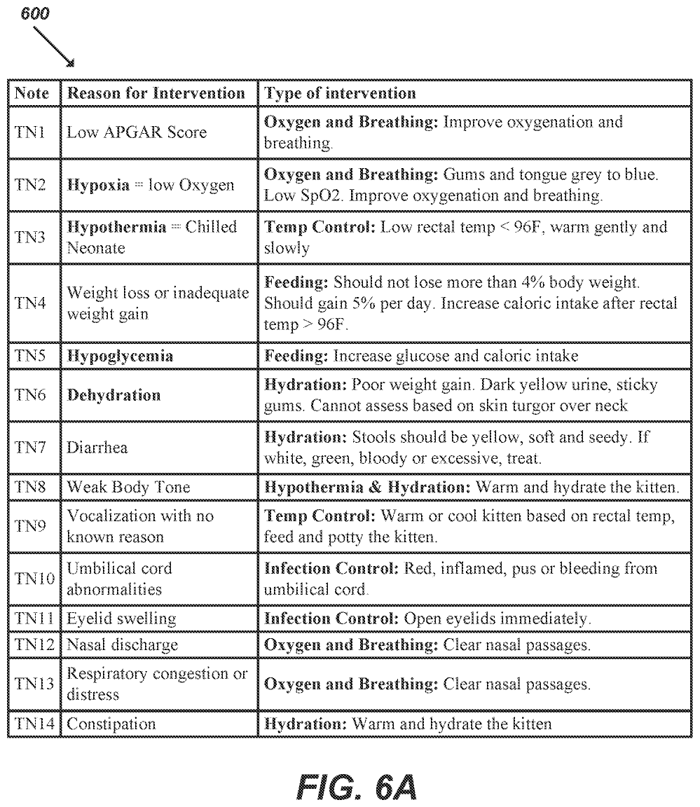

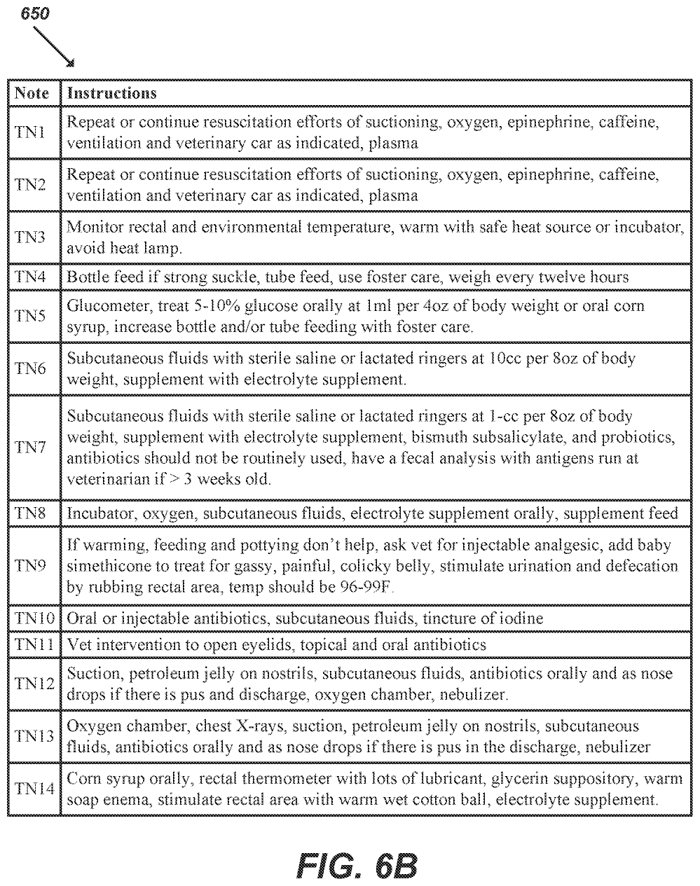

Recommendations may include specific recommendations based on individual parameter scores and/or sets of scores. For instance, each parameter may be associated with care recommendations for each score. In the kitten example, each score may have a discrete value of zero, one, or two. Each score may be mapped to a specific treatment recommendation. For example, a recommendation associated with a “nasal discharge” score of zero may be mapped to a recommendation to provide additional fluids (e.g., via sub-cutaneous injection). As another example, a “nasal discharge” score of two may be mapped to a recommendation to provide nasal antibiotics. Various example treatment mappings will be described in more detail in reference to FIG. 6A and FIG. 6B below.

Returning to FIG. 1, scores (and/or other evaluation metrics) and/or care recommendations may be provided via a resource such as user device 150, user device 180, a UI of neonate evaluator 100, and/or via other appropriate elements. Feedback may be collected via such channels as well. Such feedback may include, for instance, updates to neonate score or performance after implementation of the care recommendation(s). In some embodiments, some or all care recommendations may be implemented automatically. For instance, if neonate 110 is housed by an incubator associated with the facility 130, the incubator temperature (and/or other attributes) may be manipulated by the neonate evaluator 100 (e.g., via a set of control messages sent to the incubator from the neonate evaluator 100).

Neonate evaluator 100 may implement machine learning with respect to neonate evaluation, care recommendations, and/or other relevant attributes or parameters. For instance, if a recommended treatment is associated with more positive outcomes, machine learning models may be trained such that the treatment may be more likely to be recommended in the future. As another example, if a particular evaluation plan is associated with more positive outcomes, machine learning models may be trained such that the particular evaluation plan is more likely to be selected.

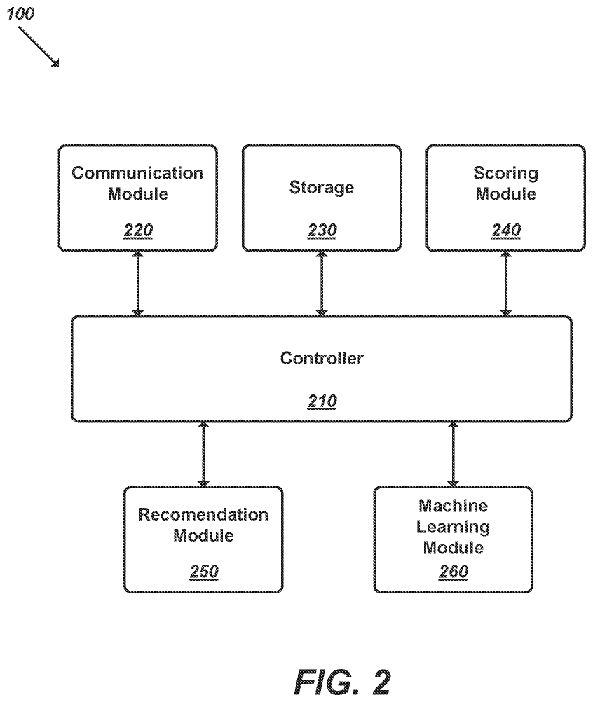

FIG. 2 illustrates a schematic block diagram of an exemplary neonate evaluator 100. As shown, neonate evaluator 100 may include a controller 210, a communication module 220, a storage 230, a scoring module 240, a recommendation module 250, and a machine learning module 260.

Controller 210 may include a processor, microcontroller, and/or other appropriate resource that is capable of executing instructions and/or otherwise processing data. Controller 210 may at least partly direct the operations of other components 220-260 of the neonate evaluator 100.

Communication module 220 may be able to communicate with other devices or systems across various wired, wireless, network, and/or other communication channels. For instance, communication module 220 may be able to communicate with user device 150 across a Bluetooth channel. As another example, communication module 220 may be able to communicate with user device 180 over the Internet. Communication module 220 may include various converters, interfaces, physical connectors, and/or other components that may allow the neonate evaluator 100 to interact with sensors 160.

Storage 230 may be an electronic device that is able to store instructions and/or data. Storage 230 may provide access to remote storages or data services. Information such as profiles, models, measurements, etc. 190 may be stored, updated, retrieved, and/or otherwise manipulated via storage 230.

Scoring module 240 may receive evaluation plans and measurements and generate various health scores or metrics. For instance, an evaluation plan may be associated with a listing of expected measurements, a score mapping for each measurement in the listing, a weighting for each measurement score, and/or other relevant information. Scoring module 240 may generate an overall health score based on each parameter listed in the evaluation plan. Scoring module 240 may normalize and/or filter received measurements, as appropriate.

Recommendation module 250 may receive score information from scoring module 240 and match the score information to various available care recommendations. Such matching may be based on specific parameter results (e.g., a score of zero on a bodyweight measurement may be associated with care including additional nourishment) and/or combinations of parameter results (e.g., a score of zero or one on a set of parameters may be associated with a care recommendation). Machine learning models may be used by some embodiments of the recommendation module 250 to identify relevant care recommendations.

Machine learning module 260 may implement various machine learning algorithms or features. For instance, machine learning module 260 may train machine learning models based on feedback such as neonate performance over time or some other documented outcomes.

One of ordinary skill in the art will recognize that neonate evaluator 100 may be implemented in various appropriate ways without departing from the scope of the disclosure. For instance, neonate evaluator 100 may include other modules than those shown (e.g., a UI module that generates and/or provides UI features such as evaluation instructions, scores or evaluation results, care recommendations, etc.).

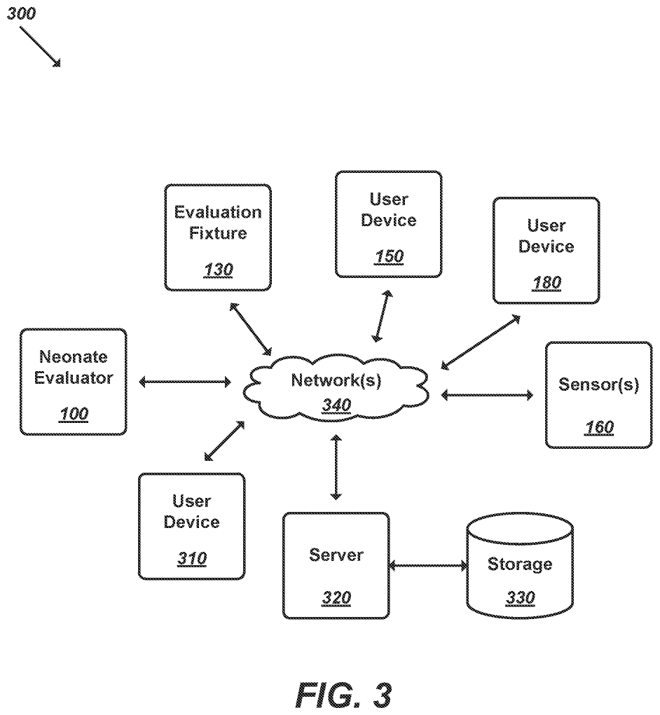

FIG. 3 illustrates a schematic block diagram of an exemplary environment 300. As shown, environment 300 may include neonate evaluator 100, evaluation facility 130, user devices 150 and 180, sensors 160, user device 310, server 320, storage 330, and networks 340.

User device 310 may be similar to user devices 150 and 180 described above. User device 310 may be able to communicate via network 340.

Server 320 may be a computing device that is able to execute instructions and/or otherwise process data. Server 320 may be able to access storage 330. Server 320 may be able to communicate via network 340.

Storage 330 may be, include, and/or utilize, one or more electronic devices or components able to store data and/or instructions. In some embodiments, storage 330 may be accessible via network 340 (e.g., via an application programming interface (API)).

Server 320 and/or storage 330 may interact with multiple neonate evaluators 100 to analyze performance and update various profiles, machine learning models, etc.

Networks 340 may include various local and/or distributed networks, such as Wi-Fi, Ethernet, the Internet, cellular networks, etc. Networks 340 may include other communication pathways, such as wired connections, local wireless channels (e.g., Bluetooth), and/or other available communication pathways or channels.

One of ordinary skill in the art will recognize that environment 300 may be implemented in various different ways without departing from the scope of the disclosure. For instance, various additional components may be included (e.g., additional servers or user devices) and/or some listed components may be eliminated or excluded (e.g., server 320).

FIG. 4 illustrates a data structure diagram of exemplary data structures 410-440 used by the neonate evaluator 100. In this example, data structures include a neonate structure 410, a sensor structure 420, a score structure 430, and a measurement structure 440.

Neonate structure 410 may include elements such as a neonate identifier, sensor identifier(s), score identifier(s), measurement identifier(s), profile data, and/or other relevant information or references. The neonate identifier may be a unique identifier that is associated with a particular neonate 110 or 120 (e.g., a unique serial number). The sensor identifiers may indicate unique sensor identifiers that are associated with the neonate and that identify associated sensor structures 420. For example, a neonate may have a microchip implant that transmits an RFID. As another example, a neonate may be associated with a wearable device (e.g., a location sensor, a heart rate monitor, etc.). Each score identifier may indicate a unique reference to a score element 430 associated with the neonate. Each measurement identifier may indicate a unique reference to a measurement element 440 associated with the neonate. Profile data may include information such as demographic information related to the neonate (e.g., animal type, breed, date of birth, ancestral information, listings of related animals such as siblings, etc.). Profile information may include information associated with a related facility, fixture, etc.

Sensor structure 420 may include elements such as a sensor identifier, sensor type, calibration information, neonate identifier(s), profile data, and/or other relevant information or references. For instance, a sensor element 420 may refer to an associated facility element (not shown). The sensor identifier may be a unique identifier that is associated with a particular sensor 160. In some cases, portions of the sensor identifier may include or provide information related to the sensor (e.g., type, output range, input range, etc.). The sensor type may indicate type or sub-type information. For instance, the sensor type element may map to a listing (e.g., a lookup table) of sensor type identifiers and sensor types (e.g., thermometer, scale, camera, microphone, humidity, etc.). Sub-type information may differentiate, for example, an infrared thermometer from a rectal thermometer or an infrared camera from a two-dimensional color camera or a three-dimensional camera. Calibration information may include, for instance, tuning information, information related to performance of the associated sensor across environmental conditions (e.g., temperature, humidity, elevation, etc.), and/or other information related to sensor performance. The neonate identifier(s) may refer to various neonate elements 410, and the associated neonates 110 or 120. Profile data may include information such as manufacturer, model, dates of calibration, and/or other relevant information.

Score structure 430 may include elements such as a score identifier, neonate identifier(s), measurement identifier(s), profile data, and/or other relevant information or references. The score identifier may be a unique identifier that is associated with a particular evaluation score. In some cases, portions of the score identifier may include or provide information related to the score (e.g., evaluation plan type, date generated, etc.). The neonate identifier(s) may refer to various neonate elements 410, and the associated neonates 110 or 120. The measurement identifier(s) may refer to various measurement elements 440. Profile data may include information such as score type, range of score, calculated score, score weighting, evaluation plan attributes or parameters, and/or other relevant information related to a score.

Measurement structure 440 may include elements such as a measurement identifier, sensor identifier, neonate identifier, profile data, and/or other relevant information or references. The measurement identifier may be a unique identifier that is associated with a particular measurement or set of measurements. In some cases, portions of the measurement identifier may include or provide information related to the measurement (e.g., sensor type, date generated, etc.). The sensor identifier(s) may refer to a set of sensor elements 420 for the set of sensors 160 associated with the measurement. The neonate identifier may refer to a neonate element 410 for the neonate 110 or 120 associated with the measurement. Profile data may include information such as measurement type, sensor type, raw value, environmental data (e.g., temperature, humidity, atmospheric pressure, etc.), and/or other relevant information related to a measurement.

One of ordinary skill in the art will recognize that different embodiments may utilize various different data structures and/or elements than those described above. For instance, some embodiments of neonate evaluator 100 may utilize a “facility” data structure that may include elements such as a unique facility identifier, facility type, references to associated sensors 420, neonates 410, scores 430, measurements 440, equipment listings, listings of associated users, etc. As another example, some embodiments of neonate evaluator 100 may utilize a “user” data structure that includes elements such as a unique user identifier, user type (e.g., technician, veterinarian, breeder, etc.), profile data, etc.

FIG. 5 illustrates a lookup table 500 associated with an exemplary evaluation plan utilized by the neonate evaluator 100. This example may be associated with evaluation of kittens, while different embodiments may utilize various different lookup tables associated with different types of neonates. In this example, the lookup table 500 includes a listing of evaluation parameters, a score mapping for each evaluation parameter, and potential care recommendations based on the score. The various care recommendations, or “treatment notes” (TNs) will be described in more detail in reference to FIG. 6A and FIG. 6B below.

Returning to FIG. 5, in this example the first evaluation parameter is the activity, pulse, grimace, appearance, and respiration (APGAR) score. Each factor of the APGAR score may range from zero to two points, resulting in a total score of zero to ten points. APGAR score may be determined automatically based on captured data (e.g., images, sensor readings, etc.), user inputs (e.g., a veterinarian or other practitioner may enter a score via a UI or similar element), and/or other relevant sources of data. Table 1 below lists an example APGAR scoring system that may be used by some embodiments of the neonate evaluator 100.

TABLE 1

| Indicator | | 0 Points | 1 Point | 2 Points |
|---|---|---|---|---|
| A | Activity | Absent | Flexed limbs | Active |
| | (muscle tone) | | | |
| P | Pulse | Absent | <100 bpm | >100 bpm |
| G | Grimace | Floppy | Minimal response | Prompt response |
| | (reflex | | to stimulation | to stimulation |
| | irritability) | | | |
| A | Appearance | Blue or | Pink body with blue | Pink |
| | (skin color) | pale | extremities | |
| R | Respiration | Absent | Slow and irregular | Vigorous cry |

The resulting total APGAR score may be mapped to a value from zero to two in this example. In this example, the score mappings are all discrete values. Different embodiments may utilize various different score mappings, such as rational numbers, percentages, ratios, and/or other appropriate mappings. In this example, a low APGAR score may be associated with TN 1 and/or TN 2 as shown.

As shown, the various other evaluation parameters may be mapped to a score from zero to two and be associated with one or more care recommendations for lower scores (e.g., scores less than two). Different embodiments may include different listings of evaluation parameters, different score mappings, and/or different associated care recommendations.

Each of the various evaluation parameters may be mapped to a score and a total or overall score may be generated based on the evaluation parameter scores. In this example, there are thirteen evaluation parameters, resulting in a range of overall scores from zero to twenty-six. The overall scores may be mapped to a care urgency parameter and/or other care recommendations. For example, an overall score of zero to six may indicate that the neonate needs immediate and urgent in-person veterinary care. Continuing the example, an overall score from seven to fourteen may indicate that the neonate merits veterinary intervention when next available. Further continuing the example, an overall score from fifteen to twenty-six may indicate that no veterinary intervention is needed, or that minimal intervention such as a telephonic of electronic care may be appropriate. Within each range, a lower score may indicate that more care is needed and/or that such care is needed more urgently.

FIG. 6A illustrates a lookup table 600 associated with exemplary care information utilized by the neonate evaluator 100. FIG. 6B illustrates a lookup table 650 associated with exemplary care recommendations utilized by the neonate evaluator 100. FIG. 6A and FIG. 6B may continue the example of FIG. 5. The example of FIG. 6A and FIG. 6B may be associated with evaluation of kittens, while different embodiments may utilize various different lookup tables associated with different types of neonates. In this example, the lookup tables 600 and 650 includes a listing of TNs, a reason for intervention, a type of intervention, and treatment instructions.

Of special note in this example are the “four Hs”: hypothermia, hypoxemia, hypovolemia (dehydration), and hypoglycemia, as indicated by bold type. Such conditions may be prioritized for treatment and/or otherwise be highlighted to resources such as care providers or practitioners such as veterinarians.

Some embodiments of the neonate evaluator 100 may provide care instructions associated with each TN. For instance, instructions related to feeding kittens may indicate that foster care and/or tube feeding may be appropriate for a particular neonate. Such instructions may indicate that kittens can take in more volume via suckling than tube feeding. The instructions may indicate that if a kitten is too weak to adequately suckle on a bottle, tube feeding may be safer and/or preferable. The instructions may indicate that a three-and-one-half Fr to five Fr feeding tube should be used. Instructions may indicate that sponge or eyedropper feeding should not be implemented due to risk of aspiration. Continuing the kitten example, the instructions may indicate that the so-called “five Ps” (premeasure tube from tip of nose to the last rib, pinch for vocalization before feeding, pass with chin down, pass down one side-preferably the left, and prewarm kitten and subcutaneous formula) of safely tube feeding newborn kittens should be used.

One of ordinary skill in the art will recognize that the lookup tables and mappings described above were presented for exemplary purposes only and that different embodiments may utilize various different lookup tables with various different arrangements and/or elements. For example, other types of neonates (e.g., puppies) may be associated with different sets of lookup tables. As another example, some embodiments may include different sets of lookup tables for other neonate attributes than type (e.g., breed).

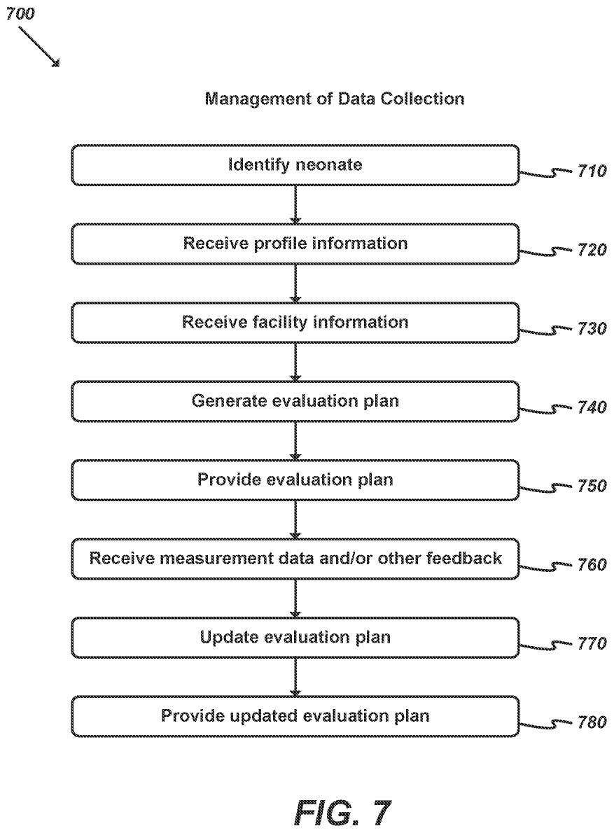

FIG. 7 illustrates an example process 700 for managing data collection for neonate evaluation. The process may present an evaluation plan that may include, for example, a listing of evaluation types to be performed. Received data may be mapped to the listing of evaluation types such that the evaluation plan may be used to generate a score or metric. The process may be performed when a neonate is evaluated. In some embodiments, process 700 may be performed by neonate evaluator 100.

As shown, process 700 may include identifying (at 710) a neonate under evaluation. Neonates may be identified in various appropriate ways. For instance, if a neonate 110 is associated with an element such as an implanted RFID tag, a sensor 160 such as an RFID reader may be able to determine the RFID and match to the associated neonate 110 (e.g., using a lookup table). As another example, a camera 160 may capture an image of the neonate 110 and the image may be compared to a database of images to identify the neonate 110. As another example, a practitioner may enter identifying information (e.g., name, serial number, etc.) for the neonate via a UI of neonate evaluator 100 or via a resource such as user device 150.

Process 700 may include receiving (at 720) profile information related to the identified neonate. For example, the neonate ID may be utilized to identify a neonate 110 via a lookup table or similar resource associated with a listing of neonate structures 410. If no profile is available (e.g., for a newborn), a new profile may be generated and/or a default profile may be selected (e.g., a generic kitten profile). Profile information may include information such as previous measurements and/or score information.

The process may include receiving (at 730) facility information for a facility associated with the neonate evaluation. Facility information may be received via a resource such as a facility structure, where the facility ID may be indicated by a resource such as the neonate structure 410. Facility information may include, for example, type of facility, available sensors and/or equipment, listings of associated neonates, listing of associated practitioners or other users, and/or other relevant information.

As shown, process 700 may include generating (at 740) an evaluation plan. An evaluation plan may be generated in various appropriate ways, via various appropriate resources. For example, based on the neonate profile information, a neonate type (e.g., kitten, puppy, etc.) and/or other appropriate attributes (e.g., breed, age, etc.) may be determined. A resource such as a lookup table may be used to identify a relevant evaluation plan template based on the neonate attributes. As one example, an evaluation plan template based on table 500 may be used for generating an evaluation plan for a kitten. The evaluation plan may include a listing of evaluation parameters to be measured or determined. The evaluation plan may be associated with various score mappings and/or care recommendations. The evaluation plan may include instructions or indications (e.g., the evaluation plan may indicate a measurement, type of sensor or instrument used to collect the measurement, range of expected values, and/or other relevant information).

Process 700 may include providing (at 750) the evaluation plan. The evaluation plan may be provided to a user 140 such as a caregiver, breeder, or practitioner via a resource such as user device 150 or a UI provided by neonate evaluator 100. The listing of evaluation parameters and/or other relevant information may be provided to the user 140 such that the user 140 may perform the evaluation. In some cases, the evaluation plan may be utilized by the neonate evaluator 100 to automatically engage or activate various sensors 160 such that neonate information may be collected. A UI associated with the evaluation plan may include, for instance, a listing of parameters, sets of inputs for entering and/or displaying measured values, a resulting score or score mapping, care recommendations for low scoring parameters, and/or other appropriate information (e.g., instructions for performing a measurement or evaluation, example images, etc.).

The process may include receiving (at 760) measurement data and/or other feedback. Data may be received directly from elements such as sensors 160 and/or via a resource such as a UI provided by neonate evaluator 100 and/or user device 150. Other feedback may include, for instance, commentary or notes received from users 140, previously implemented care recommendations, etc. In some cases, measurement data for associated neonates may be received or collected. For instance, the weight of a newborn may be compared to the weight of other newborns from the same litter in addition to, or in place of, generic weight limits based on neonate type.

As shown, process 700 may include updating (at 770) the evaluation plan. Depending on the received measurement data and/or other feedback, the evaluation plan may be updated. The evaluation plan may include an order of evaluation and/or may include conditional or branched elements. For example, if a first measurement is outside an expected range, additional measurements may be indicated and/or a different instrument or sensor 160 may be recommended or engaged. As another example, the order of evaluation may be updated based on the measured values or mapped scores.

Process 700 may include providing (at 780) the updated evaluation plan. The updated evaluation plan may be provided to a user 140 such as a caregiver, breeder, or practitioner via a resource such as user device 150 or a UI provided by neonate evaluator 100. Operations 770-780 may be performed iteratively, until the evaluation plan is completed (e.g., a measurement or other evaluation data is received for each evaluation parameter) or terminated (e.g., a user 140 may indicate that necessary equipment or time is not available to complete the evaluation plan).

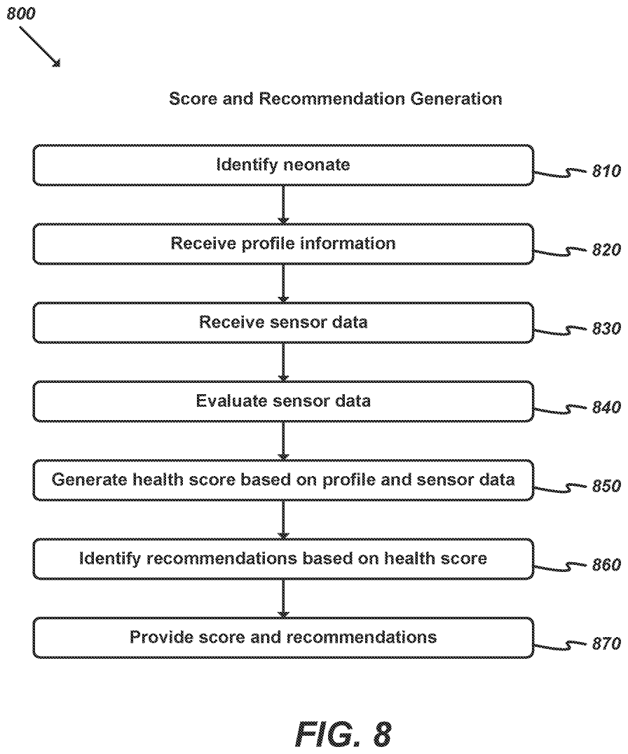

FIG. 8 illustrates an example process 800 for generating a health score and care recommendations. The process may receive the evaluation plan and measured parameters (or “evaluation results”) and generate a health score and/or care recommendations based on the score mapping of each parameter and/or the overall score. The process may be performed whenever an evaluation plan is implemented (e.g., when all parameters have been received or the evaluation plan is terminated or otherwise indicated as being completed). In some embodiments, process 800 may be performed by neonate evaluator 100.

As shown, process 800 may include identifying (at 810) a neonate. Neonates may be identified in various appropriate ways. For instance, a neonate ID may be associated with the evaluation results and the neonate ID may be used to retrieve the neonate structure 410.

Process 800 may include receiving (at 820) profile information. Profile information may be received from a resource such as storage 330. The evaluation results may include other relevant information such as the facility ID, practitioner and/or user IDs, sensor IDs, measurement IDs, etc. The various relevant profiles may be identified via the unique identifiers using resources such as lookup tables. Profile information may include information such as previous measurements and/or score information. In some embodiments, profile information may include information related to siblings (e.g., by providing a listing of neonate IDs), other litters from the same dam and/or sire, information related to other ancestors, etc. Profile information may include listings of neonate IDs associated with neonates from other litters of the same type, breed, and/or other attributes.

The process may include receiving (at 830) sensor data. Sensor data may include any measurements or evaluation data associated with the evaluation results. The data may be identified via unique measurement IDs or other appropriate ways (e.g., via a listing of elements associated with the evaluation results).

As shown, process 800 may include evaluating (at 840) sensor data. The measured or entered data may be evaluated in various appropriate ways. For instance, captured image data may be analyzed to determine various characteristics associated with the evaluation plan (e.g., activity level, color, etc.). In some cases, sensor data may be normalized or scaled based on various relevant factors, such as sensor profile information received via an element such as sensor structure 420 indicating calibration data for a particular sensor, where the calibration data may be used to normalize measurements received via the sensor.

Process 800 may include generating (at 850) a health score based on the profile and sensor data. Generation of a health score may include utilization of a resource such as lookup table 500 to map the various measured values to a score for each parameter. The elements of the evaluation plan may be summed together to generate an overall health score. If parameter measurements are missing or otherwise unavailable, default values may be used and/or the overall score may be scaled based on the number and/or type of measured parameters and unavailable measurements. For example, if half of the measurements are unavailable or unusable, the overall score may be doubled such that the output range matches the evaluation plan. In some cases, a score may be associated with a disclaimer or other limitation (e.g., “not all measurements able to be collected or completed”, “sensor data out of range”, etc.). As another example, if parameter measurements are missing or unavailable, a previous measured value may be used, if appropriate (e.g., a bodyweight measurement from the same day may be used for the evaluation if a current measurement is not available).

In some embodiments, the health score may include information related to other associated neonates (e.g., previous litters of the same parent(s), other litters of the same breed, etc.). The health score may compare attributes of neonates associated with a particular dam or sire to neonates associated with other dams and/or sires.

The process may include identifying (at 860) recommendations based on the health score(s) and/or overall health score. Recommendations may be identified using a resource such as lookup table 500. In that example, each parameter may be associated with care recommendations if the parameter score is lower than a minimum threshold (where that minimum threshold is two in most of these examples). The overall score may similarly be associated with care recommendations and/or care urgency as described above.

As shown, process 800 may include providing (at 870) the health score and recommendations. The health score(s) and care recommendations may be provided via a UI or other appropriate resource (e.g., by saving the score(s) and recommendations to an evaluation report or similar element that may include the evaluation plan, measured values, profile identifiers, and/or other relevant information).

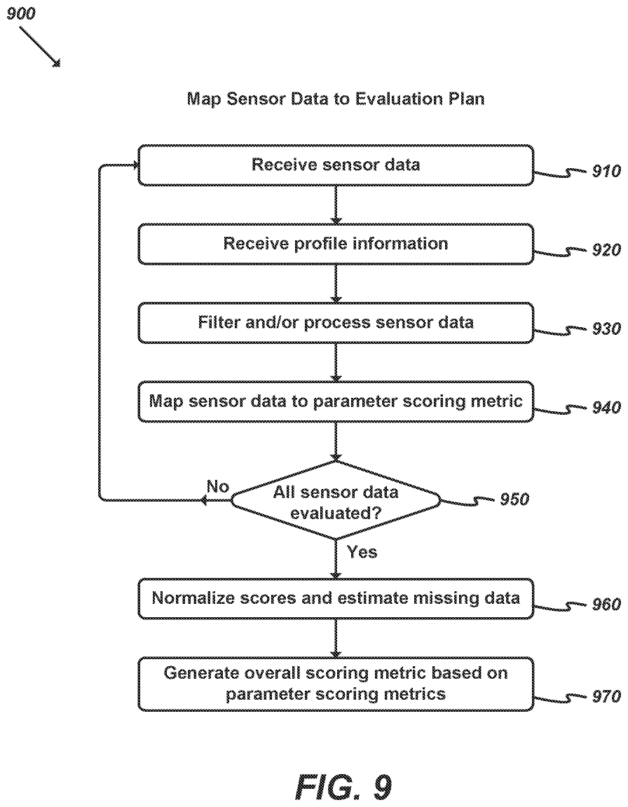

FIG. 9 illustrates an example process 900 for mapping sensor data to evaluation parameters when generating a health score. The process may match received sensor data to evaluation plan parameters as an evaluation is performed. The process may be performed when a neonate evaluation is initiated, for instance as part of operation 760 described above. In some embodiments, process 900 may be performed by neonate evaluator 100.

As shown, process 900 may include receiving (at 910) sensor data. Sensor data may be received by neonate evaluator 100 from resources such as sensors 160, user devices 150, storage 330, etc. Sensor data may be received in various appropriate formats across various appropriate channels.

Process 900 may include receiving (at 920) profile information associated with the received sensor data. Profile information may be received from a resource such as storage 330. Sensor profiles may be identified via the unique identifiers using resources such as lookup tables of sensor structures 420. Sensor profiles may include information such as sensor type, calibration data, certification information (e.g., a date the associated sensor was tested or otherwise evaluated), output range, etc. If received sensor data is not associated with, or able to be associated with, a unique sensor identifier, default or generic profiles may be used.

The process may include filtering and/or otherwise processing (at 930) the received sensor data. For example, measured data may be normalized based on sensor calibration data, sensor output range, and/or other relevant factors (e.g., previously measured data). As another example, if multiple of a particular type of sensor are available, some values may be discarded or filtered. For instance, if multiple temperature sensors are distributed about an evaluation facility 130, any readings that are not within a matching tolerance of other readings may be discarded or otherwise manipulated (e.g., by scaling such readings).

As shown, process 900 may include mapping (at 940) the sensor data to a parameter scoring metric. Such mapping may be performed in various appropriate ways. For instance, if a sensor profile indicates a type of measurement (e.g., body temperature, heart rate, etc.), the type information may be used to match the received measurements to an evaluation parameter of the same type. As another example, sensor data may be associated with various units (e.g., kilograms, pounds, etc.) that may be associated with a particular evaluation parameter (e.g., bodyweight) such that the evaluation parameter may be determined based on the measurement units. As another example, some collected information type (e.g., captured image data) may be mapped to one or more evaluation parameters (e.g., APGAR appearance, nasal discharge, eyelid swelling, etc.). As still another example, collected audio information may be mapped to a parameter such as vocalization or respiratory congestion if the received information is relevant to such evaluation parameters (e.g., audio information that can be identified as including breathing sounds may be mapped to respiratory congestion while audio information that can be identified as including screaming or whining may be mapped to vocalization.

Process 900 may include determining (at 950) whether all sensor data has been evaluated. Such a determination may be made in various appropriate ways. For instance, the listing of evaluation parameters associated with an evaluation plan may be compared to the received measurement mappings to determine whether all evaluation parameters have been mapped to at least one received measurement. As another example, a listing of each received sensor measurement may be compiled (and/or updated as measurements are received) and evaluated to determine whether each received measurement has been mapped to an evaluation parameter.

If the process determines (at 950) that all sensor data has not been evaluated, the process may repeat operations 910-950 until the process determines (at 950) that all sensor data has been evaluated.

If process 900 determines (at 950) that all sensor data has been evaluated, the process may include normalizing (at 960) scores and/or estimating missing data. Normalization of scores may be based on information such as a sensor profile, neonate profile, practitioner profile, facility profile, etc. and/or associated entities (e.g., neonate scores may be normalized based on measured values associated with littermates). As an example, a score based on inputs received from a practitioner may be normalized based on comparisons between evaluation data associated with a particular practitioner and evaluation data associated with other practitioners (e.g., if the particular practitioner has given an average score of “one” for APGAR appearance while other practitioners give an average score of “two” for the same neonates, the score of the particular practitioner may be normalized such that a score of one is mapped to a score of two). Normalization may include normalization based on availability of measurements relative to evaluation parameters (e.g., an overall score may be doubled if only half of the evaluation parameters were received).

Estimation of missing data may include, for instance, averaging of associated or relevant scores related to other evaluation parameters. For example, if urine color information is not available, the score may be estimated based on fecal character information and/or based on an average value of available parameter scores. As another example, a practitioner may indicate that a general “appearance” has a score from zero to two (or similar value or mapping) and that score may be applied to any missing data related to, for instance, weight gain/weight loss, eyelid swelling, nasal discharge, etc. that might be associated with a general appearance score as perceived by a practitioner.

In some cases, normalizing or estimating of data may be at least partly based on previously collected measurements and/or previously calculated scores.

As shown, process 900 may include generating (at 970) an overall scoring metric based on the parameter scoring metrics. The overall scoring metric may be generated in various appropriate ways. For instance, the individual scores may be summed to generate an overall score in some embodiments. In some embodiments, the score may be weighted or otherwise scaled to generate an overall score. In some embodiments each parameter score may be mapped to a value associated with an overall score.

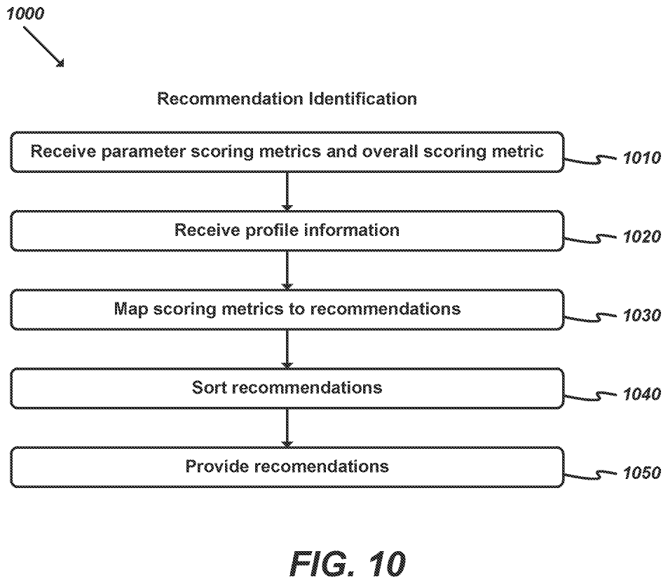

FIG. 10 illustrates an example process 1000 for identifying relevant health care recommendations based on the health score information. The process may identify relevant recommendations based on individual evaluation parameter scores and/or combined metrics (e.g., an overall health score). The process may be performed when a health score is generated. In some embodiments, process 1000 may be performed by neonate evaluator 100.

As shown, process 1000 may include receiving (at 1010) parameter scoring metrics and an overall scoring metric. Such scoring metrics may be received in various appropriate ways. For instance, an evaluation results data structure may be received from a resource such as storage 330. In some cases, scoring information may be received in real-time (e.g., during an evaluation) as sensor measurements are received and mapped to scores. In some embodiments, historical score information may also be received.

Process 1000 may include receiving (at 1020) profile information. Profile information may be received from a resource such as storage 330. The evaluation results may include relevant information in addition to the health score information, such as a facility ID, practitioner and/or user IDs, sensor IDs, measurement IDs, etc. The various relevant profiles may be identified via the unique identifiers using resources such as lookup tables.

The process may include mapping (at 1030) the scoring metrics to a set of recommendations. Recommendations may be identified using a resource such as lookup table 500. In that example, each parameter may be associated with care recommendations if the parameter score is lower than a minimum threshold. The overall score may similarly be associated with care recommendations and/or care urgency as described above.

As shown, process 1000 may include sorting (at 1040) the recommendations. Recommendations may be sorted based on various relevant criteria, such as associated parameter score (e.g., lower scores may be placed higher in a listing of recommendations than higher scores). In some cases, an evaluation plan may include an ordered list of evaluation parameters such that the recommendations may be sorted based on the order of associated evaluation parameters. As another example, the evaluation plan may include an ordered list of treatment recommendations such that the recommendations may be sorted based on the specified order.

In some cases, the recommendations may be sorted based on a comparison to historical scores or measurements. For example, if an evaluation parameter has shown improvement since a previous measurement, the treatment recommendation may be continued and/or prioritized. As another example, if an evaluation parameter has not shown improvement, a different treatment recommendation may be identified and/or prioritized.

Process 1000 may include providing (at 1050) the recommendations. The care recommendations may be provided via a UI or other appropriate resource (e.g., by saving the recommendations to a treatment report or similar element that may include the evaluation plan, measured values, profile identifiers, and/or other relevant information such as historical data).

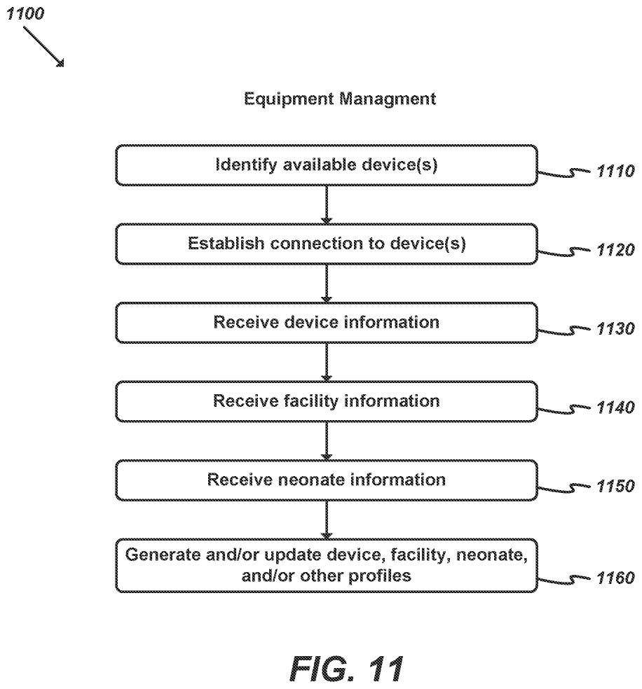

FIG. 11 illustrates an example process 1100 for managing equipment used to evaluate neonates. The process may identify equipment associated with a facility and map the equipment to relevant profiles or elements. The process may be performed whenever the neonate evaluator 100 connects to a device or component. In some embodiments, process 1100 may be performed by neonate evaluator 100.

As shown, process 1100 may include identifying (at 1110) available devices. Identification of available device may include, for instance, scanning for beacon signals or availability for pairing. As another example, a listing of devices may be received from a user 140 or a resource such as storage 330.

Process 1100 may include establishing (at 1120) a connection to each of the available devices. Such connections may be established in various appropriate ways across various appropriate paths, depending on the capabilities and/or other attributes of the available devices. For example, the neonate evaluator 100 may be able to connect to local devices across a Bluetooth or other wireless channel. As another example, the neonate evaluator 100 may be able to connect to various devices across one or more networks, such as network 340.

The process may include receiving (at 1130) device information. Device information may include information such as device type (e.g., user device, sensor, etc.), device identifier (e.g., serial number, unique name, etc.), sensor type (e.g., camera, thermometer, scale, etc.), and/or other relevant information (e.g., output type, output range, calibration information, etc.). Device information may be received across the established connection(s) and/or via a resource such as a UI. Device information may be received via a resource such as a device profile associated with a unique ID.

As shown, process 1100 may include receiving (at 1140) facility information. Facility information may include information such as facility type (e.g., veterinary office, kennel, breeder, etc.), facility location, facility attributes, listings of associated devices (e.g., sensors), etc. Facility information may be received via a resource such as a facility profile with associated unique ID.

Process 1100 may include receiving (at 1150) neonate information. If the equipment is associated with a particular neonate (e.g., a wearable or implanted device) or set of neonates (e.g., a camera associated with a kennel), information such as the neonate IDs, neonate type, etc. may be collected.

The process may include generating and/or updating (at 1160) device, facility, neonate, and/or other informational profiles. Based on the received information, and/or evaluation of existing profile information, profiles may be updated or generated based on the received information. For example, if a new piece of equipment is associated with an existing facility, the associated facility profile may be updated to include a reference to the new piece of equipment. As another example, if a device is implanted into a neonate, the neonate profile may be updated to include a reference to the device (and/or the device profile may be updated to include a reference to the neonate).

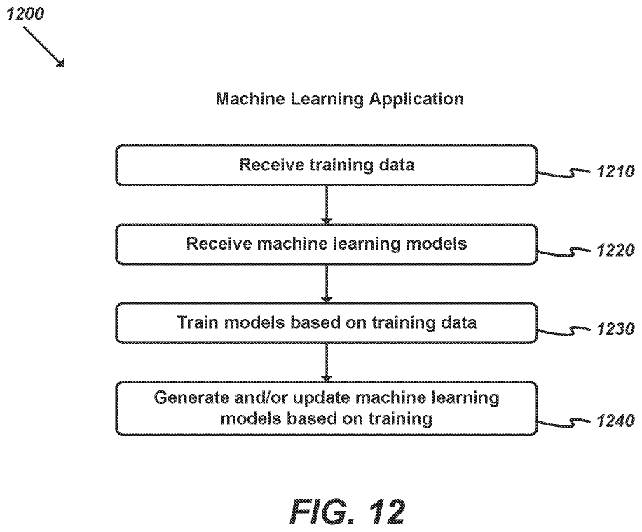

FIG. 12 illustrates an example process 1200 for applying machine learning to neonate evaluation and care recommendations. The process may evaluate training data to generate and/or update machine learning models applied to neonate evaluation and/or care. The process may be performed whenever training data becomes available, at regular interval, and/or under other appropriate conditions. In some embodiments, process 1200 may be performed by neonate evaluator 100.

As shown, process 1200 may include receiving (at 1210) training data. Training data may be received from various appropriate resources across various appropriate channels. For instance, a practitioner may upload measurement or care data via a UI of some embodiments. As another example, as evaluations are performed, the evaluation information may be supplied as training data for machine learning. Training data may include data such as feedback from users related to evaluation, care recommendations, etc.

Process 1200 may include receiving (at 1220) machine learning models. Machine learning models may be received from a resource such as storage 330.

The process may include training (at 1230) the received machine learning models based on the training data. Training may utilize or implement various machine learning algorithms, as appropriate. For instance, care recommendations associated with positive outcomes may be more likely to be recommended under similar conditions while care recommendations associated with negative outcomes may be less likely to be recommended. As another example, if practitioners provide positive feedback regarding a particular care recommendation, that care recommendation may be more likely to be recommended under similar circumstances, while care recommendations associated with negative feedback may be less likely to be recommended, regardless of the success or failure of such recommendations. For instance, if practitioners indicate that some recommendation is too difficult to implement, the recommendation may be less likely to be recommended.

As shown, process 1200 may include generating and/or updating (at 1240) the machine learning models based on the training. Based on the model training, the machine models may be updated and stored for future use.

One of ordinary skill in the art will recognize that processes 700-1200 may be implemented in various different ways without departing from the scope of the disclosure. For instance, the elements may be implemented in a different order than shown. As another example, some embodiments may include additional elements or omit various listed elements. Elements or sets of elements may be performed iteratively and/or based on satisfaction of some performance criteria. Non-dependent elements may be performed in parallel. Elements or sets of elements may be performed continuously and/or at regular intervals.

The processes and modules described above may be at least partially implemented as software processes that may be specified as one or more sets of instructions recorded on a non-transitory storage medium. These instructions may be executed by one or more computational element(s) (e.g., microprocessors, microcontrollers, digital signal processors (DSPs), application-specific integrated circuits (ASICs), field programmable gate arrays (FPGAs), other processors, etc.) that may be included in various appropriate devices in order to perform actions specified by the instructions.

As used herein, the terms “computer-readable medium” and “non-transitory storage medium” are entirely restricted to tangible, physical objects that store information in a form that is readable by electronic devices.

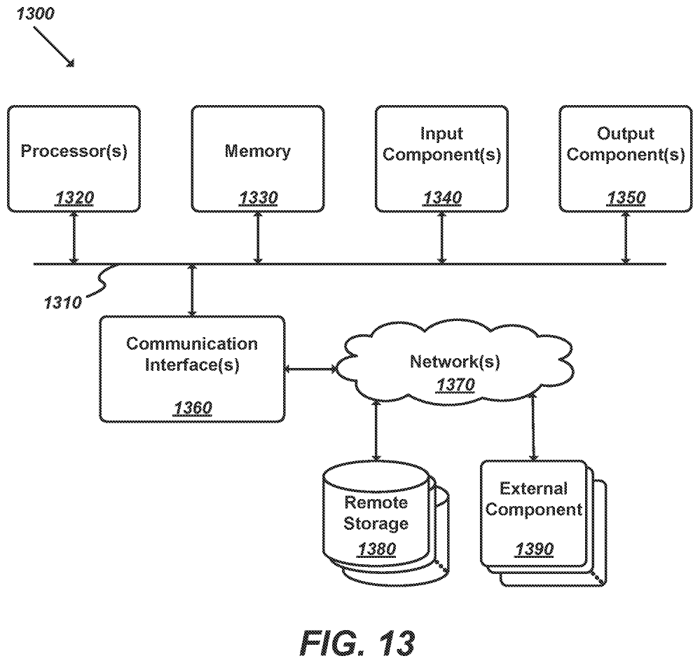

FIG. 13 illustrates a schematic block diagram of an exemplary device (or system or devices) 1300 used to implement some embodiments. For example, the systems, devices, components, and/or operations described above in reference to FIG. 1, FIG. 2, and FIG. 3 may be at least partially implemented using device 1300. As another example, the processes described in reference to FIG. 7, FIG. 8, FIG. 9, FIG. 10, FIG. 11, and FIG. 12 may be at least partially implemented using device 1300.

Device 1300 may be implemented using various appropriate elements and/or sub-devices. For instance, device 1300 may be implemented using one or more personal computers (PCs), servers, mobile devices (e.g., smartphones), tablet devices, wearable devices, and/or any other appropriate devices. The various devices may work alone (e.g., device 1300 may be implemented as a single smartphone) or in conjunction (e.g., some components of the device 1300 may be provided by a mobile device while other components are provided by a server).

As shown, device 1300 may include at least one communication bus 1310, one or more processors 1320, memory 1330, input components 1340, output components 1350, and one or more communication interfaces 1360.

Bus 1310 may include various communication pathways that allow communication among the components of device 1300. Processor 1320 may include a processor, microprocessor, microcontroller, DSP, logic circuitry, and/or other appropriate processing components that may be able to interpret and execute instructions and/or otherwise manipulate data. Memory 1330 may include dynamic and/or non-volatile memory structures and/or devices that may store data and/or instructions for use by other components of device 1300. Such a memory device 1330 may include space within a single physical memory device or spread across multiple physical memory devices.

Input components 1340 may include elements that allow a user to communicate information to the computer system and/or manipulate various operations of the system. The input components may include keyboards, cursor control devices, audio input devices and/or video input devices, touchscreens, motion sensors, etc. Output components 1350 may include displays, touchscreens, audio elements such as speakers, indicators such as light-emitting diodes (LEDs), printers, haptic or other sensory elements, etc. Some or all of the input and/or output components may be wirelessly or optically connected to the device 1300.

Device 1300 may include one or more communication interfaces 1360 that are able to connect to one or more networks 1370 or other communication pathways. For example, device 1300 may be coupled to a web server on the Internet such that a web browser executing on device 1300 may interact with the web server as a user interacts with an interface that operates in the web browser. Device 1300 may be able to access one or more remote storages 1380 and one or more external components 1390 through the communication interface 1360 and network 1370. The communication interface(s) 1360 may include one or more application programming interfaces (APIs) that may allow the device 1300 to access remote systems and/or storages and also may allow remote systems and/or storages to access device 1300 (or elements thereof).

It should be recognized by one of ordinary skill in the art that any or all of the components of computer system 1300 may be used in conjunction with some embodiments. Moreover, one of ordinary skill in the art will appreciate that many other system configurations may also be used in conjunction with some embodiments or components of some embodiments.

In addition, while the examples shown may illustrate many individual modules as separate elements, one of ordinary skill in the art would recognize that these modules may be combined into a single functional block or element. One of ordinary skill in the art would also recognize that a single module may be divided into multiple modules.

Device 1300 may perform various operations in response to processor 1320 executing software instructions stored in a computer-readable medium, such as memory 1330. Such operations may include manipulations of the output components 1350 (e.g., display of information, haptic feedback, audio outputs, etc.), communication interface 1360 (e.g., establishing a communication channel with another device or component, sending and/or receiving sets of messages, etc.), and/or other components of device 1300.

The software instructions may be read into memory 1330 from another computer-readable medium or from another device. The software instructions stored in memory 1330 may cause processor 1320 to perform processes described herein. Alternatively, hardwired circuitry and/or dedicated components (e.g., logic circuitry, ASICs, FPGAs, etc.) may be used in place of or in combination with software instructions to implement processes described herein. Thus, implementations described herein are not limited to any specific combination of hardware circuitry and software.

The actual software code or specialized control hardware used to implement an embodiment is not limiting of the embodiment. Thus, the operation and behavior of the embodiment has been described without reference to the specific software code, it being understood that software and control hardware may be implemented based on the description herein.

While certain connections or devices are shown, in practice additional, fewer, or different connections or devices may be used. Furthermore, while various devices and networks are shown separately, in practice the functionality of multiple devices may be provided by a single device or the functionality of one device may be provided by multiple devices. In addition, multiple instantiations of the illustrated networks may be included in a single network, or a particular network may include multiple networks. While some devices are shown as communicating with a network, some such devices may be incorporated, in whole or in part, as a part of the network.

Some implementations are described herein in conjunction with thresholds. To the extent that the term “greater than” (or similar terms) is used herein to describe a relationship of a value to a threshold, it is to be understood that the term “greater than or equal to” (or similar terms) could be similarly contemplated, even if not explicitly stated. Similarly, to the extent that the term “less than” (or similar terms) is used herein to describe a relationship of a value to a threshold, it is to be understood that the term “less than or equal to” (or similar terms) could be similarly contemplated, even if not explicitly stated. Further, the term “satisfying,” when used in relation to a threshold, may refer to “being greater than a threshold,” “being greater than or equal to a threshold,” “being less than a threshold,” “being less than or equal to a threshold,” or other similar terms, depending on the appropriate context.

No element, act, or instruction used in the present application should be construed as critical or essential unless explicitly described as such. An instance of the use of the term “and,” as used herein, does not necessarily preclude the interpretation that the phrase “and/or” was intended in that instance. Similarly, an instance of the use of the term “or,” as used herein, does not necessarily preclude the interpretation that the phrase “and/or” was intended in that instance. Also, as used herein, the article “a” is intended to include one or more items and may be used interchangeably with the phrase “one or more.” Where only one item is intended, the terms “one,” “single,” “only,” or similar language is used. Further, the phrase “based on” is intended to mean “based, at least in part, on” unless explicitly stated otherwise.

The foregoing relates to illustrative details of exemplary embodiments and modifications may be made without departing from the scope of the disclosure. Even though particular combinations of features are recited in the claims and/or disclosed in the specification, these combinations are not intended to limit the possible implementations of the disclosure. In fact, many of these features may be combined in ways not specifically recited in the claims and/or disclosed in the specification. For instance, although each dependent claim listed below may directly depend on only one other claim, the disclosure of the possible implementations includes each dependent claim in combination with every other claim in the claim set.

We claim:

1. A device, comprising:
one or more processors configured to:
receive profile information associated with at least one neonate;
generate an evaluation plan for the at least one neonate;
receive sensor measurements associated with the evaluation plan; and
generate an evaluation metric based on the received sensor measurements.

2. The device of claim 1, wherein the profile information includes a neonate type and the evaluation plan includes a listing of evaluation parameters associated with the neonate type.

3. The device of claim 2, wherein the evaluation plan includes a score mapping for each evaluation parameter in the listing of evaluation parameters.

4. The device of claim 3, wherein generating the evaluation metric comprises summing the score mappings for each evaluation parameter in the listing of evaluation parameters.

5. The device of claim 3, wherein generating the evaluation metric based on the received sensor measurements comprises identifying a particular evaluation parameter from the listing of evaluation parameters that is associated with each received sensor measurement from the received sensor measurements.

6. The device of claim 5, wherein generating the evaluation metric further comprises applying the score mapping for the particular evaluation parameter from the listing of evaluation parameters.

7. The device of claim 1, the one or more processors further configured to generate at least one care recommendation based on the evaluation metric.

8. A non-transitory computer-readable medium, storing a plurality of processor-executable instructions to:
receive profile information associated with at least one neonate;
generate an evaluation plan for the at least one neonate;
receive sensor measurements associated with the evaluation plan; and
generate an evaluation metric based on the received sensor measurements.

9. The non-transitory computer-readable medium of claim 8, wherein the profile information includes a neonate type and the evaluation plan includes a listing of evaluation parameters associated with the neonate type.

10. The non-transitory computer-readable medium of claim 9, wherein the evaluation plan includes a score mapping for each evaluation parameter in the listing of evaluation parameters.

11. The non-transitory computer-readable medium of claim 10, wherein generating the evaluation metric comprises summing the score mappings for each evaluation parameter in the listing of evaluation parameters.

12. The non-transitory computer-readable medium of claim 10, wherein generating the evaluation metric based on the received sensor measurements comprises identifying a particular evaluation parameter from the listing of evaluation parameters that is associated with each received sensor measurement from the received sensor measurements.

13. The non-transitory computer-readable medium of claim 12, wherein generating the evaluation metric further comprises applying the score mapping for the particular evaluation parameter from the listing of evaluation parameters.

14. The non-transitory computer-readable medium of claim 8, the plurality of processor-executable instructions further to generate at least one care recommendation based on the evaluation metric.

15. A method comprising:
receiving profile information associated with at least one neonate;
generating an evaluation plan for the at least one neonate;
receiving sensor measurements associated with the evaluation plan; and
generating an evaluation metric based on the received sensor measurements.

16. The method of claim 15, wherein the profile information includes a neonate type and the evaluation plan includes a listing of evaluation parameters associated with the neonate type.

17. The method of claim 16, wherein the evaluation plan includes a score mapping for each evaluation parameter in the listing of evaluation parameters.

18. The method of claim 17, wherein generating the evaluation metric comprises summing the score mappings for each evaluation parameter in the listing of evaluation parameters.

19. The method of claim 17, wherein:
generating the evaluation metric based on the received sensor measurements comprises identifying a particular evaluation parameter from the listing of evaluation parameters that is associated with each received sensor measurement from the received sensor measurements, and
generating the evaluation metric further comprises applying the score mapping for the particular evaluation parameter from the listing of evaluation parameters.

20. The method of claim 15 further comprising generating at least one care recommendation based on the evaluation metric.
